(19) United States
(12) Patent Application Publication
(10) Pub. No.: US 2019/0110971 A1
(43) Pub. Date: Apr. 18, 2019

(54) COMPOSITIONS FOR MANAGING PLAQUE FORMATION

(71) Applicant: THE REGENTS OF THE UNIVERSITY OF CALIFORNIA, Oakland, CA (US)

(72) Inventors: Kang TING, Los Angeles, CA (US); Wenyuan SHI, Los Angeles, CA (US); Tingxi WU, Los Angeles, CA (US); Xuesong HE, Los Angeles, CA (US); Lujia CEN, Los Angeles, CA (US)

(73) Assignee: THE REGENTS OF THE UNIVERSITY OF CALIFORNIA, Oakland, CA (US)

(21) Appl. No.: 16/097,128

(22) PCT Filed: Apr. 28, 2017
(86) PCT No.: PCT/IB2017/052498

§ 371 Date: Oct. 26, 2018

Related U.S. Application Data
(60) Provisional application No. 62/329,114, filed on Apr. 28, 2016.

(51) Int. Cl. A61K 8/44; A61Q 11/00; A61K 8/60; A61K 8/21; A61K 8/43; G01N 33/50

(52) U.S. Cl. CPC A61K 8/44; A61Q 11/00; A61K 8/60; A61K 2800/92; A61K 8/43; G01N 33/5082; A61K 2800/30; A61K 8/21

(57) ABSTRACT

Orthodontic brackets promote the formation of bacterial biofilms in the human oral cavity, which lead to a variety of oral health problems, including plaque, gingivitis, and white spot lesions. Disclosed herein are methods and compositions for the preventing or inhibiting biofilm formation and associated diseases.

### CROSS-REFERENCE TO RELATED APPLICATIONS

This application claims the benefit of priority of U.S. Provisional Patent Application No. 62/329,114, filed on Apr. 28, 2016, which is hereby incorporated by reference in its entirety.

### BACKGROUND OF THE INVENTION

### 1. Field of the Invention

The present disclosure relates to the fields of dentistry, orthodontics, and oral pharmaceuticals.

### 2. Description of Related Art

The oral cavity harbors over 700 microbial species (Human Oral Microbiome Database, on worldwide web at homd.org). These bacteria adhere to surfaces of various oral tissues (e.g., tooth, oral mucosa, tongue) and form complex, multi-species biofilms, also commonly known as dental plaque. While the majority of these bacteria are commensal, some oral microbial species are pathogens responsible for various oral diseases, including dental caries and periodontitis (Hardie 1992, Patil 2013). These diseases are exacerbated by orthodontic brackets which enhance bacterial biofilm formation.

Current methods for managing bracket-induced plaques focus primarily on mechanical removal of plaque by mechanical forces or remineralization reagents to repair the damages caused by plaque accumulation. Brush-based mechanical cleaning is the primary method for removing dental plaques on native oral tissues (both teeth and gums). Studies have shown this method to be ineffective (Boyd 1983, Costa 2007, Klukowska 2011, Laher 2003). Other methods of plaque removal techniques include fluoride and chlorhexidine delivered in the form of toothpaste, mouth rinse, varnish and gel (Baygin 2013, Santamaria 2014, van der Kaaij 2015).

The current approaches have been inadequate, and plaque-induced disease remains an unsolved problem in orthodontic patients (Alexander 1991, Boke 2014, Consolaro 2013, Lucchese 2013, Tufekci 2011, van Gastel 2007, Zachrisson 1972). Novel treatments that target orthodontic apparatus-induced plaques and their associated oral diseases are needed.

### SUMMARY

Bacterial polysaccharide and polypeptide binding ligands enable bacteria to attach to surfaces in the oral cavity as the first step in biofilm formation. The compositions disclosed herein comprise sugars and/or amino acids that block polysaccharide and polypeptide binding ligands on bacteria. The compositions therefore prevent or inhibit plaque formation and buildup in various oral cavity indications. The compositions may be used to prevent or inhibit plaque formation in individuals having or wearing one or more dental appliances, which includes orthodontic brackets, at least one dental implant, dentures (partial, flexible partial, and full), an aligner, a mouthguard, a night guard, a snoring device in contact with the teeth, a dental guard, or a retainer (collectively “dental appliances”). It is contemplated that one or more of these may be excluded in an embodiment described herein. In certain embodiments, a composition or method may be used on a patient who is not wearing a dental appliance.

In some embodiments compositions and methods for prevention or inhibition of plaque formation and buildup in individuals are disclosed. In some aspects, methods of preventing or inhibiting plaque formation in an individual with orthodontic brackets are disclosed. In some embodiments, methods of preventing or inhibiting plaque formation in an individual susceptible to tooth decay, such as someone who wears a dental appliance, are disclosed. In some aspects, methods of preventing or inhibiting plaque formation in an individual having native teeth are disclosed. The methods comprise locally administering to the oral cavity of the individual a composition comprising at least one isolated amino acid and at least one sugar, in some embodiments. In some embodiments, compositions and methods for prevention or inhibition of plaque formation and buildup in individuals comprise locally administering to the oral cavity of the individual a composition consisting of at least one isolated amino acid and at least one sugar. In some embodiments, compositions and methods for prevention or inhibition of plaque formation and buildup in individuals comprise locally administering to the oral cavity of the individual a composition consisting essentially of at least one isolated amino acid and at least one sugar. In some aspects, compositions and methods for prevention or inhibition of plaque formation and buildup do not include an abrasive. In certain embodiments, a composition consists or consists essentially of 1) at least 1, 2, 3, 4, 5, 6, 7, 8, 9, or 10 (and any range derivable therein) isolated amino acid(s) or amino acid derivative(s) and 2) at least 1, 2, 3, 4, 5, 6, 7, 8, 9, or 10 sugar(s) (or any range derivable therein). In a specific embodiment, methods and compositions exclude an abrasive. In some aspects, the composition is formulated for local administration to the oral cavity and is not substantially ingested. In some aspects, the composition is a toothpaste, gel, varnish or mouthwash. Toothpastes include conventional toothpaste and baby toothpaste.

In some embodiments, the at least one isolated amino acid is an amino acid analog or an amino acid derivative. In further embodiments, the at least one isolated amino acid is selected from the group consisting of arginine, lysine, alanine, threonine, and histidine. The at least one amino acid may be a dextrorotatory (D) or a levorotatory (L) amino acid. In some embodiments, a composition for preventing or inhibiting plaque formation comprises two or more isolated amino acids. In some embodiments, a composition for preventing or inhibiting plaque formation consists of or consists essentially of two or more isolated amino acids. In additional embodiments, a composition for preventing or inhibiting plaque formation consists essentially of two or more isolated amino acids. In further embodiments, a composition for preventing or inhibiting plaque formation comprises three isolated amino acids. In specific embodiments, a composition for preventing or inhibiting plaque formation comprises the isolated amino acids L-arginine, L-alanine, and L-lysine.

In some embodiments, a composition for preventing or inhibiting plaque formation in an individual having or wearing orthodontic brackets, at least one dental implant, dentures, at least one aligner, a retainer, or native teeth comprises at least one sugar, including but not limited to acetylglucosamine, galactose, mannose, and fructose. In some embodiments, a composition for preventing or inhibiting plaque formation in an individual having or wearing orthodontic brackets, at least one dental implant, dentures, at least one aligner, a retainer, or native teeth consists of at least one sugar. In some embodiments, a composition for preventing or inhibiting plaque formation in an individual having or wearing orthodontic brackets, at least one dental implant, dentures, at least one aligner, a retainer, or native teeth consists essentially of at least one sugar. In some aspects, the composition does not include an abrasive. The at least one sugar may be a sugar analog or a sugar derivative. In some aspects, the composition further comprise a fluoride source. In further aspects, the compositon may comprise an antibacterial component, including, but not limited to chlorhexidine. In some aspects, an individual does not drink or eat within 30 minutes or more after administration of the composition. A composition for preventing or inhibiting plaque formation in an individual may further comprise one or more flavorings.

Some aspects of the present disclosure are directed towards methods for identifying compounds that inhibit one or more bacterial species that colonize in the oral cavity of an animal. An animal may be a human or non-human animal. The methods may comprise, in some aspects, subjecting an in vitro oral cavity model to conditions that mimic in vivo growth conditions of the one or more bacterial species, treating the in vitro oral cavity model with at least one compound, measuring an indicator of bacterial species population on oral cavity model, and comparing the indicator of bacterial species population on the treated oral cavity model to an indicator of bacterial species population an untreated control sample of the oral cavity model. The at least one compound may be one more more isolated amino acids, one or more sugars, or combinations thereof. The at least one compound may consist of one more more isolated amino acids, one or more sugars, or combinations thereof. The at least one compound may consist essentially of one more more isolated amino acids, one or more sugars, or combinations thereof. The at least one amino acid may be an amino acid analog or an amino acid derivative. The at least one sugar may be a sugar analog or a sugar derivative. In further embodiments, the oral cavity model is treated with a plurality of compounds selected from one or more isolated amino acids, amino acid analogs or amino acid derivatives, and one or more sugars, sugar analogs, or sugar derivatives.

Providing the at least one compound prevents or inhibits the one or more bacterial species from binding to the oral cavity model, in some aspects. In some embodiments, providing the at least one compound inhibits a polysaccharide or peptide binding ligand on a bacterial surface. In some aspects, providing a plurality of compounds results in a synergistic inhibition of plaque formation or a synergistic reduction of existing plaque. In some aspects, measuring an indicator of bacterial species population on a treated oral cavity model comprises quantifying a biofilm formation or growth of a biofilm. In some embodiments, the indicator of bacterial species population is a plaque. In further embodiments, the indicator of bacterial species population is gingivitis or white spot lesions.

In some aspects, the oral cavity model is human dental tissue. The human dental tissue may belong to a human of any age, e.g., a baby, a child, an adolescent, an adult, or an elderly person. The dental tissue may be native dental tissue, or the dental tissue may comprise at least one dental implant. The human dental tissue may further comprise orthodontic brackets attached to the human dental tissue. The oral cavity model or dental tissue may further comprise at least one aligner or a retainer. In some embodiments, the oral cavity model is a denture model. In some aspects, the oral cavity model is oral mucosa. In other embodiments, the oral cavity model is human tongue tissue. In other embodiments, the subject may be another animal with teeth, such as a dog, cat, horse, or other mammal susceptible to tooth decay.

In additional aspects, methods for identifying compounds that inhibit one or more bacterial species that colonize in the oral cavity of an animal further comprise assembling a database of compounds that inhibit one or more bacterial species from colonizing upon a human biological tissue, and using the database to predict the structures of other compounds that will inhibit or prevent growth of the bacterial species on the human tissue.

In particular aspects, a method of preventing or inhibiting plaque formation in an individual who wears a dental appliance comprises locally administering to the oral cavity of the individual a toothpaste, gel, varnish, or mouthwash composition comprising a fluoride source, chlorhexidine, N-acetylglucosamine or another sugar, and at least one isolated amino acid selected from the group consisting of L-arginine, D-lysine, L-lysine, D-alanine, L-alanine, and D-threonine, wherein the composition is not substantially ingested. In some embodiments, the toothpaste, gel, varnish, or mouthwash composition does not include an abrasive. In certain embodiments, a composition comprises a fluoride source, chlorhexidine, N-acetylglucosamine or another sugar, and 1, 2, 3, 4, 5, or 6 isolated amino acid(s) selected from the group consisting of L-arginine, D-lysine, L-lysine, D-alanine, L-alanine, and D-threonine,

In some embodiments, a composition comprises at least one isolated amino acid. In other embodiments, a composition comprises at least one sugar. In other embodiments, a composition comprises at least one sugar and at least one isolated amino acid. In further embodiments, the composition does not include an abrasive.

In some embodiments, a sugar, an isolated amino acid, or a composition containing at least one or both are administered to an individual that is susceptible to plaque formation. In some aspects, the composition does not include an abrasive. In some embodiments, the individual wears orthodontic brackets, while in other embodiments, the individual wears a dental appliance that may or may not be removeable. If the appliance is removable, the composition may be administered when the individual is wearing the dental appliance, it may be administered to the individual by first applying the composition to the appliance, which is then placed in the individual's mouth, or the composition may be administered when the individual is not wearing a dental appliance. In the last scenario, the sugar(s), isolated amino acid(s) or the combination may be administered to the individual within 24 hours of having had or placing the dental appliance in the individual's oral cavity. In certain embodiments, the sugar(s), isolated amino acid(s) or the combination are applied when food is not ingested.

It is contemplated that in some embodiments, the composition is not intended to be ingested. In certain embodiments, the composition is not substantially ingested, which means that less than 25% of the composition that is placed in the mouth does not enter the digestive system. In some embodiments, the composition is expectorated or spit out after being administered to the individual. In some cases, the composition is gargled or swiched prior to being expectorated. In other embodiments, the composition is applied directly to the teeth either with a brush, other mechanical dental device, or by placing on the teeth a dental appliance having the composition on the dental appliance already. It is contemplated that in some embodiments, the composition does not include an abrasive, such as calcium carbonate. An abrasive is a particle which can induce wear on a tooth surface by scratching, gouging, chiseling, or other mechanical means, upon coming into frictional contact with the tooth surface.

As discussed above, compositions and methods for preventing or inhibiting plaque formation in an individual with orthodontic brackets (called braces by laypersons) can be similarly used with an individual who develops a similar buildup of plaque due to other orthodontic devices such as removable dental aligners (with or without bumps or attachments) or dental retainers.

As used herein, the phrases “treating and/or preventing” or “treatment and/or prevention” includes the administration of the compositions, compounds or agents of the invention to prevent or delay the onset of the symptoms, complications, or biochemical indicia of a disease, alleviating or ameliorating the symptoms or arresting or inhibiting further development of the disease, condition, or disorder (e.g., plaque formation). “Treating and/or preventing” further refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a dentist. Accordingly, the phrase “treating and/or preventing” includes the administration of the therapeutic agents of the disclosure to prevent or delay, to alleviate, or to arrest or inhibit development of the symptoms or conditions associated with biofilm formation and growth.

A “therapeutically effective amount” of a substance./molecule may vary according to factors such as the disease state, age, sex, and weight of the individual, and the ability of the substance/molecule to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated,such as a reduction in the number of bacteria.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. The transitional phrase “consisting of” excludes any element, step, or ingredient not specified in the claim. A claim which depends from a claim which “consists of” the recited elements or steps cannot add an element or step. When the phrase “consists of” appears in a clause of the body of a claim, rather than immediately following the preamble, it limits only the element set forth in that clause; other elements are not excluded from the claim as a whole. The transitional phrase “consisting essentially of” limits the scope of a claim to the specified materials or steps “and those that do not materially affect the basic characteristic(s)” of the claimed invention. In certain embodiments, the only active ingredients are an amino acid(s) and a sugar(s).

Throughout this application, the term “about” is used to indicate that a value includes the standard deviation of error for the device and/or method being employed to determine the value.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

As used herein, in the specification, “a” or “an” may mean one or more, unless clearly indicated otherwise. As used herein, in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. (a) Is an image of teeth to which orthodontic brackets are adhered. Teeth have been stained with plaque disclosing agent to reveal plaque formation (darkened areas on teeth). (b) Is an image of white spot lesions on teeth after orthodontic braces have been removed. (c) Is an image of gingivitis, i.e., inflammation in the gum tissue, in a patient with orthodontic braces.

FIG. 2 is a drawing depicting bracket-induced plaque formation. The formation of plaque involves complex microbial adherence and biofilm formation that are heavily depended on polypeptides or polysaccharides on bacterial surfaces.

FIGS. 3A-3B. FIG. 3A Is a diagram depicting the first step in bacterial biofilm formation. Bacterial surfaces comprise polypeptides and polypeptides that recognize and bind to tooth surface monosaccharides and amino acid ligands, respectively. FIG. 3B is a diagram depicting a mechanism of inhibition of biofilm formation. Administration of monosaccharides or amino acids inhibit extracellular polysaccharide and polypeptide binding to a tooth surface.

FIGS. 4A-4B. FIG. 4A Left: Hydroxyapatite (HA) disc. Right: Hydroxyapatite (HA) disc with adhered orthodontic bracket. FIG. 4B Plaque microbial compositions in original saliva samples and in vitro grown saliva derived plaque on discs analyzed by denature gradient gel. The microbial composition of saliva-derived plaques grown in vitro on discs was almost identical to original human saliva samples, demonstrating that the screening assay closely mimics in vivo conditions.

FIG. 5A-5C. FIG. 5A is an example in vitro screening result comparing inhibitor-treated (right) and untreated (left) HA discs with adhered orthodontic brackets. The discs have been stained with a plaque disclosing agent. The untreated disc on the left includes dark spots corresponding to plaque formation. FIG. 5B is a bar graph quantifying effects on biofilm growth for a range of compounds. FIG. 5C depicts results of an experiment comparing the in vitro efficacy of the chemical mixture (OS-001) against bracket-induced plaque formation.

FIGS. 6A-6E. FIG. 6A is a photograph that depicts the effect of chemical mixture OS-001 on biofilm formation. FIG. 6B is a graph that quantifies the effect of chemical mixture OS-001 on biofilm formation. FIG. 6C is a graph that illustrates the effect of chemical mixture OS-001 on the detachment of existing biofilm. FIG. 6D is a graph that illustrates the effect of chemical mixture OS-001 on the planktonic growth of a microbial community. FIG. 6E is a graph depicting the results of a cytotoxicity assay using Human Oral Keratinocytes-16B cells.

### DETAILED DESCRIPTION

A compound or compounds of the disclosure can be administered at a unit dose ranging from about 50 ppm to about 50,000 ppm based on a toothpaste, mouth rinse, varnish, gel, or other vehicle. That is, the compound or compounds may be administered at 50, 100, 200, 500, 1,000, 2,000, 5,000, 10,000, 50,000 ppm, or any amount therebetween. The administered concentration of the compound or compounds may be adjusted to be outside of the 50 ppm to 50,000 ppm range in order to attain a beneficial therapeutic endpoint.

A compound or compounds of the disclosure can be administered at a unit dose less than about 15 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of compound (e.g., about 4.4.times.1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of compound per kg of bodyweight. Particularl embodiments have dosages that are less than 2, 1, or 0.1 mg/kg for the sugar(s) or isolated amino acid(s) of body weight.

Delivery of a compound of the disclosure (amino acid and/or sugar) can be at a dosage on the order of about 0.00001 mg to about 3 mg per organ/tissue, or preferably about 0.0001-0.001 mg per organ/tissue, about 0.03-3.0 mg per organ/tissue, about 0.1-3.0 mg per organ/tissue or about 0.3-3.0 mg per organ/tissue. The dosage can be an amount effective to treat or prevent biofilm formation and associated diseases. In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In certain embodiment, a subject is administered an initial dose, and one or more maintenance doses of a composition. The maintenance dose or doses are generally lower than the initial dose, e.g., one-half less of the initial dose. A maintenance regimen can include treating the subject with a dose or doses ranging from 0.01 mg/kg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in conditions, e.g., reduction of plaque. The dosage of the compound may either be increased in the event the patient does not respond significantly to current dosage levels, or the dose may be decreased if a reduction of biofilm or reduction of associated diseases including plaque and gingivitis observed, or if undesired side effects are observed.

The effective dose can be administered in a single dose or in two or more doses, as desired or considered appropriate under the specific circumstances. Following successful treatment, it may be desirable to have the patient undergo maintenance therapy to prevent the recurrence of the disease state, wherein the compound of the invention is administered in maintenance doses, ranging from 0.01 mg per kg to 100 mg per kg of body weight (see U.S. Pat. No. 6,107,094).

The “effective amount” of the compound is an amount sufficient to be effective in treating or preventing a disorder or to regulate a physiological condition in humans. The concentration or amount of sugar and/or amino acid agent administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a composition of the invention can include a single treatment or, preferably, can include a series of treatments. It will also be appreciated that the effective dosage of composition for treatment may increase or decrease over the course of a particular treatment. Changes in dosage may result and become apparent from the results of diagnostic assays as described herein. For example, the subject can be monitored after administering a compound of the disclosure. Based on information from the monitoring, an additional amount of the compound can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models.

In some embodiments, a composition comprises at least one isolated amino acid. In other embodiments, a composition comprises at least one sugar. In other embodiments, a composition comprises at least one sugar and at least one isolated amino acid. In particular embodiments, the composition is composed of about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40% v/v or w/w (and any range derivable therein) of one or more sugars, one or more isolated amino acids, or a combination of one or more sugars and one or more isolated amino acids. Alternatively, a composition contains the following amount of a sugar, a combination of sugars, an isolated amino acid, a combination of isolated amino acids, or a combination of sugars and amino acids: about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or any range derivable therein. In addition, a subject may be administered or applied to teeth in an amount listed above in a single unit dose or a treatment regimen.

In compositions containing both at least one isolated amino acid and at least one sugar, the ratio of the amino acid(s) to the sugar(s) may be about 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1 or more, or any range derivable therein.

Pharmaceutical Preparations

In one aspect, the methods disclosed herein can include the administration of pharmaceutical compositions and formulations comprising at least one amino acid and/or at least one sugar capable of preventing or inhibiting plaque formation in an individual with orthodontic brackets.

In certain embodiments, the compositions are formulated with a pharmaceutically acceptable carrier. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The active agents can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring agents, preservatives and antioxidants can also be present in the compositions.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, toothpastes, varnishes, powders, emulsions, lyophilized powders, sprays, creams, lotions, controlled release formulations, etc. In some aspects, the formulation does not comprise an abrasive component.

In certain embodiments, the pharmaceutical compounds and formulations are lyophilized. Stable lyophilized formulations comprising an amino acid and/or sugar can be made by lyophilizing a solution comprising the active ingredient(s) and a bulking agent, e.g., mannitol, trehalose, raffinose, or mixtures thereof. A process for preparing a stable lyophilized formulation can include lyophilizing a solution about 2.5 mg/mL active composition, about 15 mg/mL bulking agent, about 19 mg/mL NaCl, and a sodium citrate buffer having a pH greater than 5.5 but less than 6.5. See, e.g., U.S. 20040028670.

The formulations can be administered for prophylactic and/or therapeutic treatments. In certain embodiments, for therapeutic applications, compositions are administered to a subject with orthodontic brackets for preventing or treating plaque formation; this can be called a therapeutically effective amount. For example, in certain embodiments, pharmaceutical compositions are administered in an amount sufficient to treat plaque formation.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

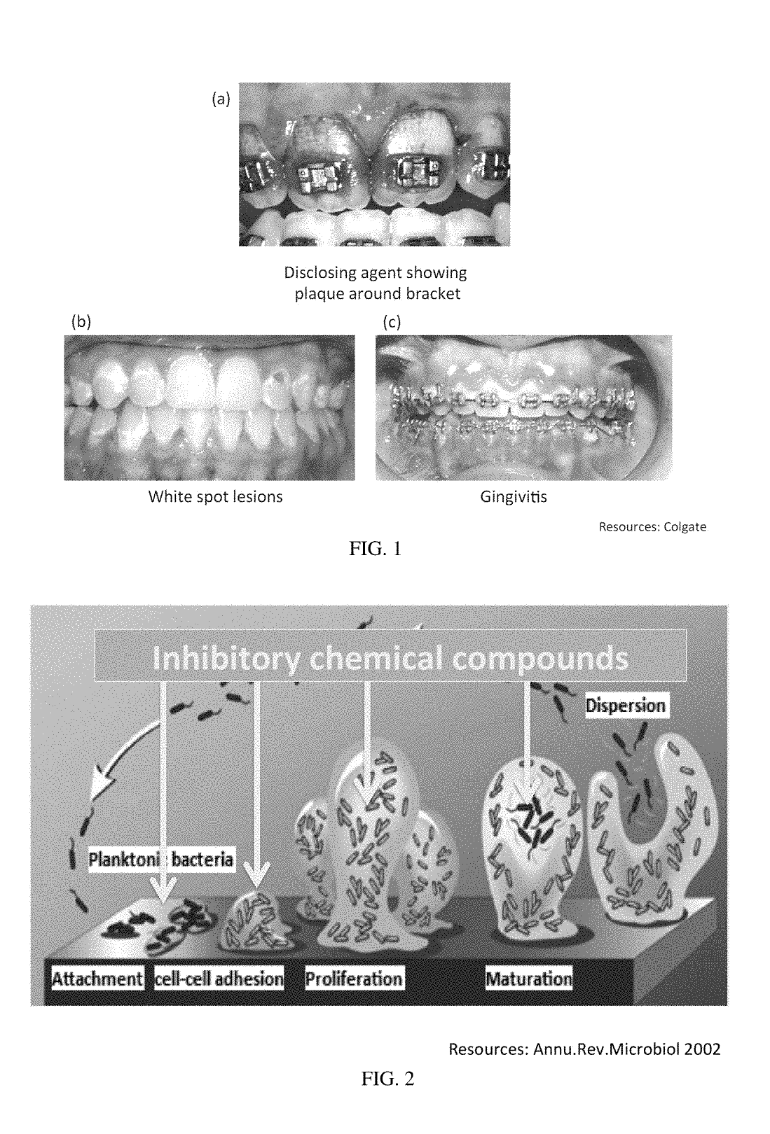

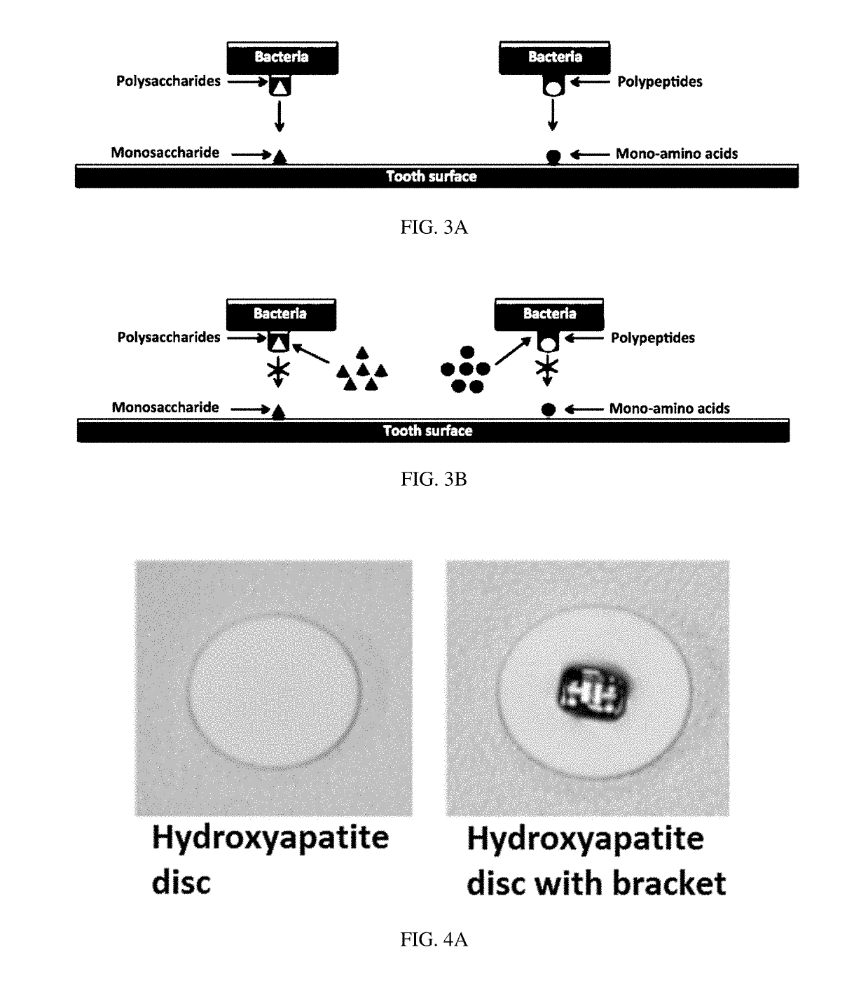

Orthodontic treatment is popular among juveniles and adults (AAO 2012, Christopherson EA 2009). As part of orthodontic treatment, brackets are adhered to tooth enamel surfaces to provide the holding bases for wires. One of the key unintended side-effects for fixed brackets on teeth surfaces is the alteration of tooth surface topology from smooth surfaces to new uneven, non-smooth junctions between teeth and brackets. These non-smooth junctions encourage enhanced microbial biofilm formation, as documented by various previous studies (Freitas AO 2014, Sukontapatipark W 2001) and illustrated in FIG. 1 (a). As mentioned above, removing dental plaque on smooth teeth surfaces by brushing is often inadequate; adding brackets and wires on these surfaces further hampers effective oral hygiene (Boyd RL 1983, Costa MR 2007, Klukowska M 2011, Laher A 2003). Consequently, it is a well-known clinical fact that a large portion of patients undergoing orthodontic treatments suffer from oral health problems associated with bracket-induced plaque formation, including white spot lesions and gingivitis, illustrated in FIG. 1 (b) and (c).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Scientific Data

To examine compounds' inhibitory effects against bracket-induced plaque formation, an in vitro bracket-induced biofilm model system was developed to structurally and functionally mimic the in vivo clinical reality. Over 200+ amino acids and sugar monomers and analogs were screened using this model system, which led to the discovery of several compounds with strong inhibitory effects against bracket-induced plaque formation. These compounds were found to be safe under laboratory safety tests and have now been formulated into prototype toothpaste with good in vitro efficacy.

### Example 2

### Development of an In Vitro Bracket-Induced Biofilm Model System

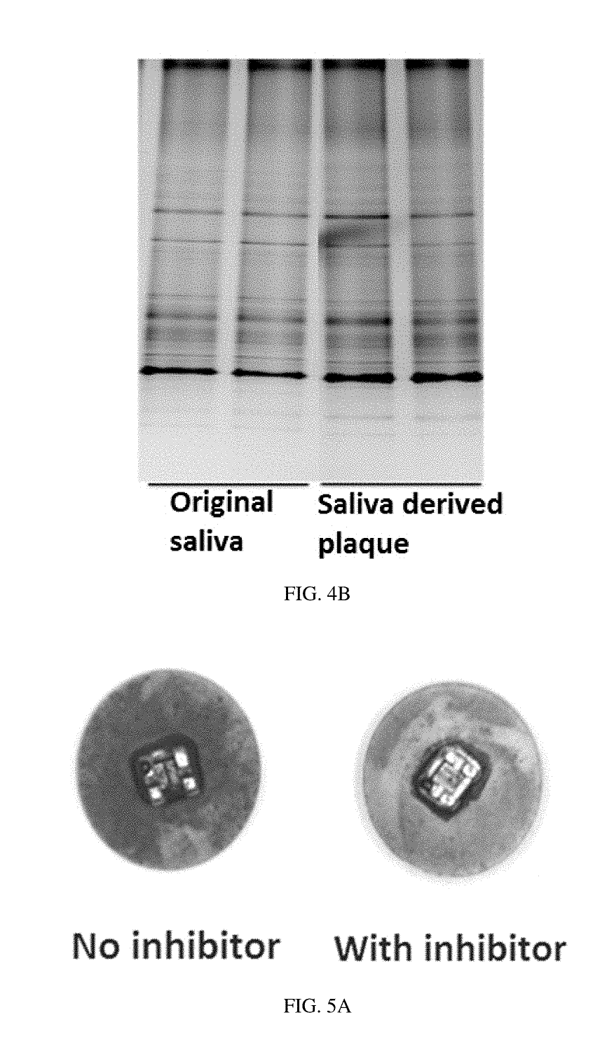

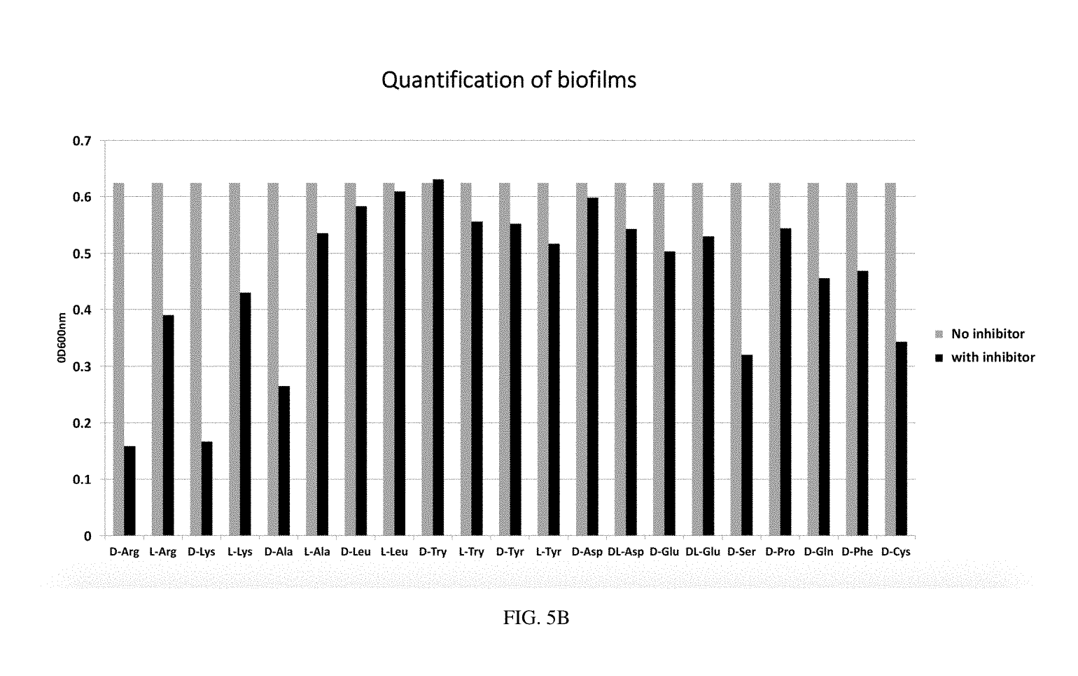

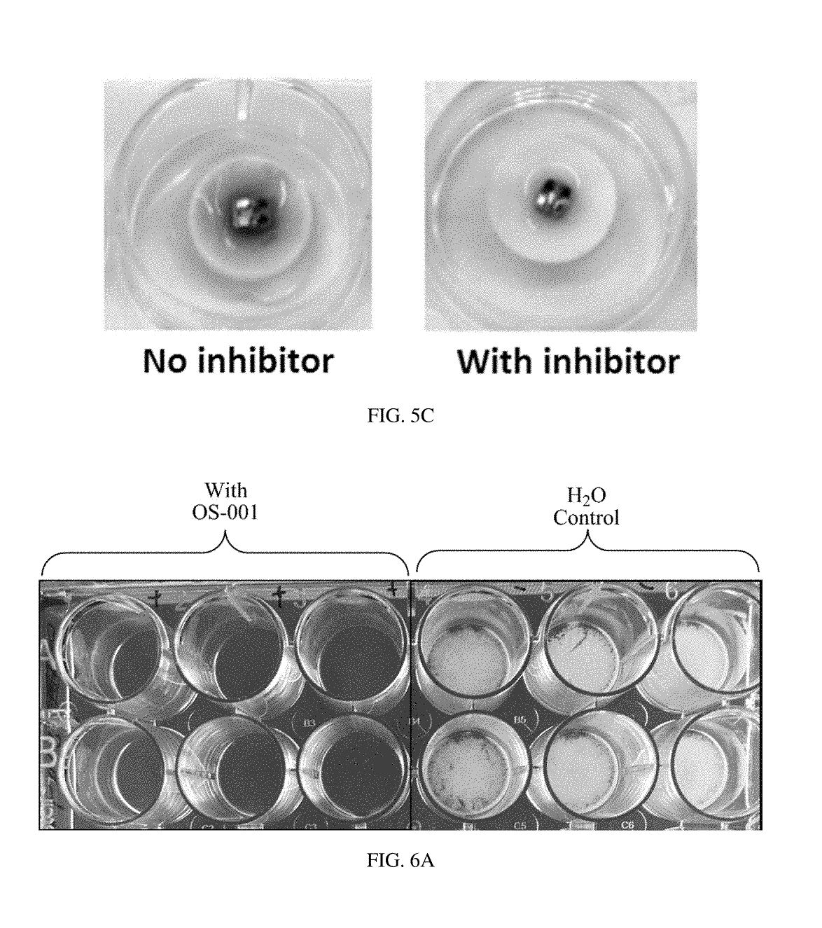

As shown in FIG. 4A, brackets were adhered to small discs (13 mm in diameter) made of hydroxyapatite (HA) as the base supporting material. These discs were then fitted into 12-well tissue culture plates for the growth of saliva-derived plaques. One unique feature of this system is use of a new culture medium developed by this laboratory (Shi-medium, Tian Y 2010) which allows the biofilm grown in vitro to be very similar to in vivo human plaque. FIGS. 5A-5C demonstrate that the microbial composition of saliva-derived plaques grown in vitro on discs was almost identical to original human saliva samples, demonstrating that this is a high-fidelity screening assay that closely mimics in vivo conditions.

### Example 3

### Identification of Compounds with Inhibitory Activity Against Bracket-Induced Plaque Formation

A mixed pool of human saliva samples was collected from 10 volunteers to develop in vitro grown plaques on the model system developed above. Amino acids and sugar monomers and analogs were selected mostly from the list of FDA Generally Recognized As Safe (GRAS) compounds (http://www.fda.gov/Food/Ingredients PackagingLabeling/GRAS/SCOGS/ default.htm). The anti-biofilm inhibitory function of these chemicals was assayed with a crystal violet assay (Sharma A 2005), as illustrated in FIG. 5A. The compounds with more than 10× inhibitory effects were further investigated. Several amino acids and sugars, including L-Arg, L-Lys, D-Lys, D-Ala, D-Thr and N-acyl-Glucosamine, showed strong inhibitory effects (FIG. 5B).

### Example 4

### A Formula with Safety and Efficacy Against Bracket-Induced Plaque Formation

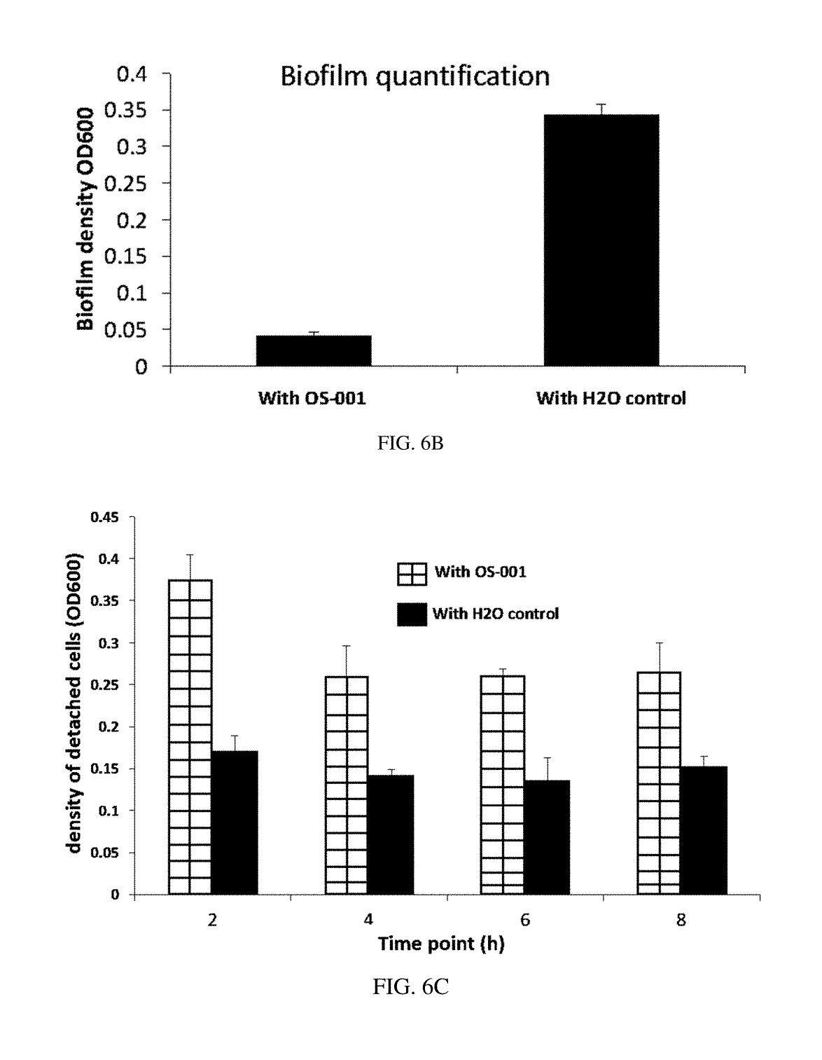

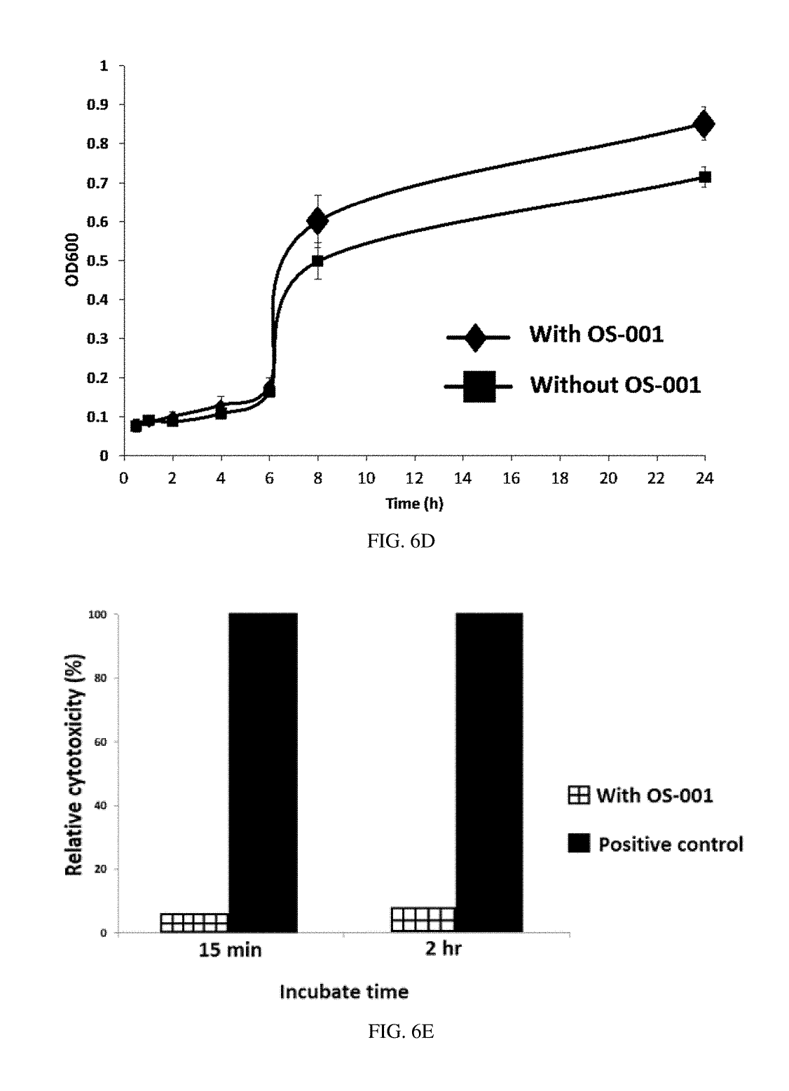

After screening provided a list of candidate compounds for further study, individual compounds were combined in different ratios to create a mixture with maximum bioactivity against bracket-induced plaque formation. This chemical mixture was then formulated into a mixture with retained bioactivity as shown in FIG. 5C. Additionally, various in vitro safety tests were conducted, including genotoxicity with Ames test (Mortelmans K 2000), cytotoxicity on T cells and B-cells CellTiterBlue or CellTiterGlo assays (Promega), and found no detectable toxicity issues. As shown in FIGS. 6A-6E, the chemical mixture (OS-001) displays strong bioactivity in inhibiting the biofilm formation of a microbial community containing hundreds of bacterial species (FIGS. 6A and 6B). OS-001 is effective in dispersing the existing multispecies biofilm (FIG. 6C), and in preventing the bracket-induced plaque formation (FIG. 5C). The formula does not have cytotoxicity effect against bacterial cells (FIG. 6D). Additionally, various in vitro safety tests were conducted, including genotoxicity with Ames test (Mortelmans K 2000), cytotoxicity on T cells and B-cells CellTiterBlue or CellTiterGlo assays (Promega), and found no detectable toxicity issues (FIG. 6E).

### Example 5

### Safety and Efficacy Studies

Two randomized, double-blind, placebo-controlled studies are disclosed. The aims of the studies are to evaluate the safety of administrating this newly formulated toothpaste in adolescent subjects, and assess the efficacy of this newly formulated toothpaste in reducing bracket-induced plaque accumulation among adolescent subjects. The subjects include 60 male and female orthodontic subjects ranging in age from 10 to 15 years old. The goal of the first study is to evaluate the safety of a newly-formulated toothpaste in adolescent subjects. The goal of the second study is to assess the efficacy of the newly-formulated toothpaste in reducing plaque accumulation among adolescent subjects.

### Example 6

### Safety Study

A cohort of 4 adolescent subjects is conducted prior to commencing enrollment of subjects. For both cohorts (safety-only and efficacy), clinic visits for subjects include Initial Visit 1 (day 0), and Follow-up Visits (Days 7 and 14). Subjects enrolled in the Adolescent Safety-Only Cohort are assessed for safety parameters only. Safety monitoring at day 0, 7 and 14 includes vital signs, intraoral assessments of hard and soft tissues, targeted physical examination, and collection of adverse events during study visits and unscheduled telephone contacts.

### Example 7

### Efficacy Study

A randomized, double-blind, placebo-controlled, safety and efficacy study in male and female orthodontic subjects 10-15 years of age is disclosed. The study includes 60 adolescent subjects (10-15 years of age).

For the efficacy study, adolescent subjects are administered study materials (active compounds disclosed herein or mixtures thereof) for 14 days. The study compares the study material administration vs. placebo. The study subjects are randomly assigned into two groups (treatment or placebo) using an interactive web response system (IWRS). Before dosing of study material, eligible subjects undergo professional dental examination, consultation and plaque examination using Trace Plaque Disclosing Liquid (DentaKit Item #: DK-R719). Quantitative plaque scores are recorded according to Quigley-Hein plaque index (Goyal CR 2005). Subjects are then be subjected to plaque removal at the dental chair and given study materials (take-home toothpaste and Oral-B toothbrush) to be used at home twice daily for 14 days. To evaluate the anti-plaque effect, study subjects are recalled to the clinic and examined for bracket-induced plaque formation using the same Plaque Disclosing Liquid (DentaKit Item #: DK-R719) protocol on days 7 and 14, respectively.

### Example 8

### Inclusion Criteria

Inclusion criteria for the adolescent safety-only cohort include males and females, 10-15 years of age, inclusive, at the time the Assent and Informed Consent Form is signed. The subjects are healthy, as determined by an investigator (in consultation with the Medical Monitor, as needed), based on medical and dental history, concurrent illnesses, laboratory results, concomitant medications, oral cavity assessment, and targeted physical examination (extra oral, head and neck) during screening. The subjects have ongoing orthodontic treatment and re wearing orthodontic brackets. The subjects are willing to refrain from using non-study dentifrice and other non-study oral care products during the study. The subjects are willing to postpone elective dental procedures (e.g., dental cleanings) between wcreening and final post-treatment visit (end of study or early termination). The subjects are willing and able to comply with oral hygiene and diet instructions (not applicable for adolescent Safety only cohort subjects). The subjects are able to understand and sign the assent and/or informed consent form prior to initiation of study procedures. The subjects are able to communicate with the Investigator/study center personnel, understand and comply with the study requirements, and willing to return for protocol-specified visits at the appointed times

### Example 9

### Exclusion Criteria

Exclusion criteria include advanced periodontal disease, and active caries lesion(s) within 30 days prior to study material administration (confirmed by dental examination and standard radiographs). Subjects presenting with white spot(s) are not excluded. Additional exclusion criteria include medical conditions (e.g., artificial heart valve, history of infective endocarditis, cardiac transplant with valvular dysfunction, congenital heart disease or total joint replacement) for which antibiotics are recommended prior to dental visits and/or procedures, suspicious or confirmed pathologic lesions of the oral cavity, current use of systemic antibiotics, topical oral antibiotics, or use of other drugs, which in the opinion of the investigator could influence the study outcome, within 30 days prior to screening. Additional exclusion criteria include participation in a clinical trial or receipt of a non-FDA approved therapy within 30 days prior to study drug administration (depending on the specifics, participation in an observational study is not necessarily excluded), and the presence of any condition or concurrent illness, which in the opinion of the Investigator, would compromise normal immune function (e.g., diabetes, rheumatoid arthritis, lupus, liver disease, organ transplant, etc.), interfere with the use of study dentifrice and oral care products, or interfere with the ability to comply with study requirements, or jeopardize the safety of the subject or the validity of the study results.

### Example 10

### Blinding/Unblinding and Data Analysis

The study is conducted in a randomized, double-blinded, placebo-controlled manner. To ensure blinding, active and placebo materials are packaged in the same manner and IWRS is be used for random assignment. Subjects are blinded to study treatment. The investigator, study staff/clinicians and the sponsor's assigned team members (e.g., the Clinical Monitor and the Medical Monitor) are blinded as to whether subjects are receiving active or placebo until the study is formally unblinded for data analysis purposes.

The total number of subjects for this study is approximately 64 (n=≥4 for Adolescent Safety Only Cohort; n=60 for Study), which is of sufficient power for the studies. The data provides information on the safety and efficacy of multiple administrations of OS-001 toothpaste or Placebo. There re two analysis sets: the safety analysis set and the efficacy analysis set.

Safety: All safety-related interventions, adverse events (AEs), and findings are summarized. The incidence and duration of treatment emergent AEs re summarized. All adverse events (AEs) are classified by the relationship of the event to study materials as 1) None, 2) Unlikely, 3) Possible or 4) Probable. All AEs re followed in accordance with good medical practice until resolved or fully characterized. All serious AEs (SAEs) are followed until the outcome is known or the subject's condition has stabilized.

Efficacy: The plaque accumulation index data revealed by the Trace Plaque Disclosing Liquid (DentaKit Item #: DK-R719) protocol is recorded and analyzed. Day 7 and 14 data re normalized with day 0 data among each subject. Descriptive statistics utilizing mean, standard deviation, median and range of plaque accumulation among active and placebo groups at each time point (days 0, 7 and 14) are determined. The difference between active and placebo groups and individual subjects in day 7 and 14 is compared and analyzed.

### REFERENCES

Alexander S A. Effects of orthodontic attachments on the gingival health of permanent second molars. Am J Orthod Dentofacial Ortho. 1991 100:337-340.

American Association of Orthodontists AAO Patient Census Surveys 1989-2010. Bull Am Assoc Orthod. 2012.

BayginO, Tuzuner T, Ozel M B, et al. Comparison of combined application treatment with one-visit varnish treatments in an orthodontic population. Med Oral Patol Oral Cir Bucal. 2013 18(2):e362-370.

Boke F, Gazioglu C, Akkaya S, et al. Relationship between orthodontic treatment and gingival health: A retrospective study. Eur J Dent. 2014 8(3): 373-380.

Christopherson E A, Briskie D, Inglehart M R. Objective, subjective, and self-assessment of preadolescent orthodontic treatment need—A function of age, gender, and ethnic/racial background? J Public Health Dent. 2009 69:9-17.

Consolaro A. In adults: 47.2% have periodontitis! How about in orthodontic patients? Dental Press J Orthod. 2013 18(1): 3-5.

Costa M R, Silva V C, Miqui M N, et al. Efficacy of ultrasonic, electric and manual toothbrushes in patients with fixed orthodontic appliances. Angle Orthod. 2007 77(2):361-366.

CellTiter-Glo® Luminescent

Cell Viability Assay world wide web @ promega.com/˜/media/files/resources/protocols/technical%20bulletins/0/celltiter%20glo%201uminescent%20cell%20viability%20assay%20protocol.pdf

CellTiter-Blue® Cell

Viability Assay world wide web @ promega. com/˜/media/files/resources/protocols/technical%20bulletins/101/celltiter-blue%20cell%20viability%20as say%20protocol.pdf

Freitas A O, Marquezan M, Nojima Mda C, et al. The influence of orthodontic fixed appliances on the oral microbiota: a systematic review. Dental Press J Orthod. 2014 19(2): 46-55.

Hardie J M. Oral microbiology: current concepts in the microbiology of dental caries and periodontal disease. Br Dent J. 1992 172(7): 271-278.

Klukowska M, Bader A, Erbe C, et al. Plaque levels of patients with fixed orthodontic appliances measured by digital plaque image analysis. Am J Orthod Dentofacial Orthop. 2011 139(5): e463-470.

Laher A, Kroon J, Booyens S J. Effectiveness of four manual toothbrushes in a cohort of patients undergoing fixed orthodontic treatment in an academic training hospital. SADJ. 2003 58(6): 234-237.

Lucchese A, Gherlone E. Prevalence of white-spot lesions before and during orthodontic treatment with fixed appliances. Eur J Orthod. 2013 35(5): 664-668.

Mortelmans K, Zeiger E. The Ames Salmonella/microsome mutagenicity assay. Mutat Res. 2000 455(1-2): 29-60.

Nobbs A H, Lamont R, Jenkinson H F, et al. Streptococcus Adherence and Colonization. Microbiol Mol Biol Rev. 2009 73(3): 407-450.

Patil S, Rao R S, Sanketh D S, et al. Microbial flora in oral diseases. J Contemp Dent Pract. 2013 14(6): 1202-1208.

Ren Y, Jongsma M A, Mei L, et al. Orthodontic treatment with fixed appliances and biofilm formation—a potential public health threat? Clin Oral Investig. 2014 18(7): 1711-1718.

Santamaria M Jr, Petermann K D, Vedovello S A, et al. Antimicrobial effect of Melaleuca alternifolia dental gel in orthodontic patients. Am J Orthod Dentofacial Orthop. 2014 145(2): 198-202.

Sukontapatipark W, el-Agroudi M A, Selliseth N J, et al. Bacterial colonization associated with fixed orthodontic appliances. A scanning electron microscopy study. Eur J Orthod. 2001 23(5): 475-484.

Tian Y, He X, Torralba M, et al. Using DGGE profiling to develop a novel culture medium suitable for oral microbial communities. Mol Oral Microbiol. 2010 25(5): 357-367.

Tufekci E, Dixon J S, Gunsolley J C, et al. Prevalence of white spot lesions during orthodontic treatment with fixed appliances. Angle Orthod. 2011 81(2): 206-210.

van der Kaaij N C, van der Veen M H, van der Kaaij M A, et al. A prospective, randomized placebo-controlled clinical trial on the effects of a fluoride rinse on white spot lesion development and bleeding in orthodontic patients. Eur J Oral Sci. 2015 123(3): 186-193.

van Gastel J, Quirynen M, Teughels W, et al. The relationships between malocclusion, fixed orthodontic appliances and periodontal disease. A review of the literature. Aust Orthod J. 2007 23(2):121-129.

Zachrisson S, Zachrisson B U. Gingival condition associated with orthodontic treatment. Angle Orthod. 1972 42: 26-34.

1. A method of preventing or inhibiting plaque formation in an individual comprising,
locally administering to the oral cavity of the individual a composition comprising at least one isolated amino acid and at least one sugar;
wherein the composition is not substantially ingested.

2. The method of claim 1, wherein the individual has or wears a dental appliance.

3. The method of claim 1, wherein the composition is a toothpaste, gel, varnish, or mouthwash.

4. The method of claim 1 or 3, wherein the at least one isolated amino acid is selected from the group consisting of arginine, lysine, alanine, threonine, and histidine.

5. The method of claim 4, wherein the arginine is L-arginine.

6. The method of claim 4, wherein the arginine is D-arginine.

7. The method of claim 4, wherein the lysine is D-lysine.

8. The method of claim 4, wherein the lysine is L-lysine.

9. The method of claim 4, wherein the alanine is D-alanine.

10. The method of claim 4, wherein the alanine is L-alanine.

11. The method of claim 4, wherein the threonine is D-threonine.

12. The method of claim 4, wherein the threonine is L-threonine.

13. The method of any of claims 1-11, wherein the at least one sugar is a sugar analog or a sugar derivative.

14. The method of claim 1 or 3, wherein the composition further comprises two or more isolated amino acids.

15. The method of claim 1 or 3, wherein the composition further comprises three isolated amino acids.

16. The method of either of claim 14 or 15, wherein the isolated amino acids are selected from the group consisting of arginine, lysine, alanine, threonine, and histidine.

17. The method of claim 15, wherein the three isolated amino acids are L-arginine, L-alanine, and L-lysine.

18. The method of any of claims 1-16, wherein the isolated amino acid is an amino acid analog or an amino acid derivative.

19. The method of any of claims 13-18, wherein the sugar analog or sugar derivative is N-acetylglucosamine, galactose, mannose, or fructose.

20. The method of any of claims 13-19, wherein the composition further comprises fluoride.

21. The method of any of claims 13-20, wherein the composition further comprises an antibacterial component.

22. The method of claim 21, wherein the antibacterial component is chlorhexidine.

23. The method of claim 1, where the individual does not drink or eat within 30 minutes after administration of the composition.

24. The method of claim 1, wherein the composition comprises one or more flavorings.

25. The method of claim 1, wherein the composition does not comprise an abrasive.

26. The method of claim 25, wherein the abrasive is calcium carbonate.

27. A method of preventing or inhibiting plaque formation in an individual comprising,
locally administering to the oral cavity a composition comprising at least one isolated amino acid;
wherein the composition is not substantially ingested.

28. The method of claim 27, wherein the individual has or wears a dental appliance.

29. The method of preventing or inhibiting plaque formation of claim 27, wherein the composition is a toothpaste, gel, varnish, or mouthwash.

30. The method of claim 27 or 29, wherein the at least one isolated amino acid is selected from the group consisting of arginine, lysine, alanine, threonine, and histidine.

31. The method of claim 30, wherein the arginine is L-arginine.

32. The method of claim 30, wherein the arginine is D-arginine.

33. The method of claim 30, wherein the lysine is D-lysine.

34. The method of claim 30, wherein the lysine is L-lysine.

35. The method of claim 30, wherein the alanine is D-alanine.

36. The method of claim 30, wherein the alanine is L-alanine.

37. The method of claim 30, wherein the threonine is D-threonine.

38. The method of claim 30, wherein the threonine is L-threonine.

39. The method of claim 27 or 29, wherein the composition further comprises two or more isolated amino acids.

40. The method of claim 27 or 29, wherein the composition further comprises three isolated amino acids.

41. The method of either of claim 39 or 40, wherein the isolated amino acids are selected from the group consisting of arginine, lysine, alanine, threonine, and histidine.

42. The method of claim 40, wherein the three isolated amino acids are L-arginine, L-alanine, and L-lysine.

43. The method of any of claims 27-41, wherein the isolated amino acid is an amino acid analog or an amino acid derivative.

44. The method of any of claims 27-43, wherein the composition further comprises a fluoride source.

45. The method of any of claims 27-44, wherein the composition further comprises an antibacterial component.

46. The method of claim 45, wherein the antibacterial component is chlorhexidine.

47. The method of claim 27, where the individual does not drink or eat within 30 minutes after administration of the composition.

48. The method of claim 27, wherein the composition comprises one or more flavorings.

49. The method of claim 27, wherein the composition does not comprise an abrasive.

50. The method of claim 49, wherein the abrasive is calcium carbonate.

51. A method of preventing or inhibiting plaque formation in an individual comprising,
locally administering to the oral cavity of the individual a composition comprising at least one sugar;
wherein the composition is not substantially ingested.

52. The method of claim 51, wherein the individual has or wears a dental appliance.

53. The method of claim 51, wherein the composition is a toothpaste, gel, varnish, or mouthwash.

54. The method of claim 51 or 53, wherein the at least one sugar is a sugar analog or a sugar derivative.

55. The method of any of claims 51-54, wherein the sugar analog or sugar derivative is N-acetylglucosamine, galactose, mannose, or fructose.

56. The method of any of claims 51-55, wherein the composition further comprises a fluoride source.

57. The method of any of claims 51-56, wherein the composition further comprises an antibacterial component.

58. The method of claim 57, wherein the antibacterial component is chlorhexidine.

59. The method of claim 51, where the individual does not drink or eat within 30 minutes after administration of the composition.

60. The method of claim 51, wherein the composition comprises one or more flavorings.

61. The method of claim 51, wherein the composition does not comprise an abrasive.

62. The method of claim 61, wherein the abrasive is calcium carbonate.

63. An oral composition for preventing or inhibiting plaque formation in an individual;
wherein the composition comprises at least one isolated amino acid and at least one sugar;
wherein the composition is formulated for local administration to the oral cavity; and
wherein the composition is not substantially ingested.

64. The method of claim 63, wherein the individual has or wears a dental appliance.

65. The oral composition of claim 63, wherein the oral composition is a toothpaste, gel, varnish, or mouthwash.

66. The oral composition of claim 63 or 65, wherein the at least one isolated amino acid is selected from the group consisting of arginine, lysine, alanine, threonine, and histidine.

67. The oral composition of claim 66, wherein the arginine is L-arginine.

68. The oral composition of claim 66, wherein the arginine is D-arginine.

69. The oral composition of claim 66, wherein the lysine is D-lysine.

70. The oral composition of claim 66, wherein the lysine is L-lysine.

71. The oral composition of claim 66, wherein the alanine is D-alanine.

72. The oral composition of claim 66, wherein the alanine is L-alanine.

73. The oral composition of claim 66, wherein the threonine is D-threonine.

74. The oral composition of claim 66, wherein the threonine is L-threonine.

75. The oral composition of any of claims 63-73, wherein the at least one sugar is a sugar analog or a sugar derivative.

76. The oral composition of claim 75, wherein the sugar analog or sugar derivative is N-acetylglucosamine, galactose, mannose, or fructose.

77. The oral composition of any of claims 63-76, wherein the isolated amino acid is an amino acid analog or an amino acid derivative.

78. The oral composition of any of claims 63-77, the composition further comprising two or more isolated amino acids.

79. The oral composition of any of claims 63-77, the composition further comprising three isolated amino acids.

80. The oral composition of either of claim 78 or 79, wherein the isolated amino acids are selected from the group consisting of arginine, lysine, alanine, threonine, and histidine.

81. The oral composition of claim 79, where the three isolated amino acids are L-arginine, L-alanine, and L-lysine.

82. The oral composition of any of claims 63-80, wherein the oral composition further comprises a fluoride source.

83. The oral composition of any of claims 63-82, wherein the oral composition further comprises an antibacterial component.

84. The oral composition of claim 83, wherein the antibacterial component is chlorhexidine.

85. The oral composition of claim 63, wherein the composition comprises one or more flavorings.

86. The oral composition of claim 63, wherein the composition does not comprise an abrasive.

87. The oral composition of claim 86, wherein the abrasive is calcium carbonate.

88. An oral composition for preventing or inhibiting plaque formation in an individual;
wherein the composition comprises at least one isolated amino acid;
wherein the composition is formulated for local administration to the oral cavity; and
wherein the composition is not substantially ingested.

89. The method of claim 88, wherein the individual has or is wearing orthodontic brackets, at least one dental implant, dentures, at least one aligner, a retainer, or native teeth.

90. The oral composition of claim 88, wherein the oral composition is a toothpaste, gel, varnish, or mouthwash.

91. The oral composition of either of claim 88 or 90, wherein the at least one isolated amino acid is selected from the group consisting of arginine, lysine, alanine, threonine, and histidine.

92. The oral composition of claim 91, wherein the arginine is L-arginine.

93. The oral composition of claim 91, wherein the arginine is D-arginine.

94. The oral composition of claim 91, wherein the lysine is D-lysine.

95. The oral composition of claim 91, wherein the lysine is L-lysine.

96. The oral composition of claim 91, wherein the alanine is D-alanine.

97. The oral composition of claim 91, wherein the alanine is L-alanine.

98. The oral composition of claim 91, wherein the threonine is D-threonine.

99. The oral composition of claim 91, wherein the threonine is L-threonine.

100. The oral composition of any of claims 88-98, wherein the isolated amino acid is an amino acid analog or an amino acid derivative.

101. The oral composition of either of claim 88 or 90, wherein the composition further comprises two or more isolated amino acids.

102. The oral composition of either of claim 88 or 90, wherein the composition further comprises three isolated amino acids.

103. The oral composition of either of claim 101 or 102, wherein the isolated amino acids are selected from the group consisting of arginine, lysine, alanine, threonine, and histidine.

104. The oral composition of claim 102, wherein the three isolated amino acids are L-arginine, L-alanine, and L-lysine.

105. The oral composition of any of claims 88-103, wherein the oral composition further comprises a fluoride source.

106. The oral composition of any of claims 88-105, wherein the oral composition further comprises an antibacterial component.

107. The oral composition of claim 107, wherein the antibacterial component is chlorhexidine.

108. The oral composition of claim 88, wherein the composition comprises one or more flavorings.

109. The oral composition of claim 88, wherein the composition does not comprise an abrasive.

110. The oral composition of claim 109, wherein the abrasive is calcium carbonate.

111. An oral composition for preventing or inhibiting plaque formation in an individual;
wherein the composition comprises at least one sugar;
wherein the composition is formulated for local administration to the oral cavity; and
wherein the composition is not substantially ingested.

112. The method of claim 111, wherein the individual has or is wearing orthodontic brackets, at least one dental implant, dentures, at least one aligner, a retainer, or native teeth.

113. The oral composition of claim 111, wherein the composition is a toothpaste, gel, varnish, or mouthwash.

114. The oral composition of claim 111 or 113, wherein the at least one sugar is a sugar analog or a sugar derivative.

115. The oral composition of claim 114, wherein the sugar analog or sugar derivative is N-acetylglucosamine, galactose, mannose, or fructose.

116. The oral composition of any of claims 111-115, wherein the composition further comprises a fluoride source.

117. The oral composition of any of claims 111-116, wherein the composition further comprises an antibacterial component.

118. The oral composition of claim 117, wherein the antibacterial component is chlorhexidine.

119. The oral composition of claim 111, wherein the composition comprises one or more flavorings.

120. The oral composition of claim 111, wherein the composition does not comprise an abrasive.

121. The oral composition of claim 120, wherein the abrasive is calcium carbonate.

122. A method for identifying compounds that inhibit one or more bacterial species that colonize in the oral cavity of an animal comprising
subjecting an in vitro oral cavity model to conditions that mimic in vivo growth conditions of the one or more bacterial species;
treating the in vitro oral cavity model with at least one compound;
measuring an indicator of bacterial species population on oral cavity model; and
comparing the indicator of bacterial species population on the treated oral cavity model to an indicator of bacterial species population an untreated control sample of the oral cavity model.

123. The method of claim 122, wherein the compounds are selected from isolated amino acids and sugars.

124. The method of either of claim 122 or 81, wherein the oral cavity model is human dental tissue.

125. The method of claim 124, wherein the human dental tissue further comprises orthodontic brackets attached to the human dental tissue.

126. The method of claim 124, wherein the human dental tissue comprises at least one dental implant.

127. The method of claim 124, wherein the human dental tissue further comprises at least one aligner.

128. The method of claim 124, wherein the human dental tissue further comprises a retainer.

129. The method of claim 122 or 123, wherein the oral cavity model is a denture model.

130. The method of either of claim 122 or 123, wherein the oral cavity model is oral mucosa.

131. The method of either of claim 122 or 123, wherein the oral cavity model is human tongue tissue.

132. The method of any of claims 122-131, wherein providing the at least one compound prevents or inhibits the one or more bacterial species from binding to the oral cavity model.

133. The method of any of claims 122-132, wherein providing the at least one compound inhibits a polysaccharide or peptide binding ligand on a bacterial surface.

134. The method of any of claims 122-133, further comprising treating the oral cavity model with a plurality of compounds.

135. The method of claim 134, wherein the plurality of compounds comprises at least one isolated amino acid and at least one sugar.

136. The method of claim 135, wherein the at least one isolated amino acid is an amino acid analog or an amino acid derivative.

137. The method of claim 135, wherein the at least one sugar is a sugar analog or a sugar derivative.

138. The method of any of claims 122-137, wherein measuring the indicator of bacterial species population on a treated oral cavity model comprises quantifying a biofilm formation.

139. The method of any of claims 122-137, wherein measuring the indicator of bacterial species population on a treated oral cavity model comprises quantifying a biofilm growth.

140. The method of any of claims 122-139, wherein the indicator of bacterial species population is a plaque.

141. The method of any of claims 122-139, wherein the indicator of bacterial species population is gingivitis.

142. The method of any of claims 122-141, further comprising assembling a database of compounds that inhibit one or more bacterial species from colonizing upon a human biological tissue, and using the database to predict the structure of other compounds that will inhibit or prevent growth of the bacterial species on the human tissue.

143. A method of preventing or inhibiting plaque formation in an individual having or wearing a dental appliance comprising,
locally administering to the oral cavity of the individual a composition comprising a fluoride source, chlorhexidine, N-acetylglucosamine or another sugar, and at least one isolated amino acid selected from the group consisting of L-arginine, L-lysine, D-lysine, L-alanine D-alanine, and D-threonine;
wherein the composition is a toothpaste, gel, varnish, or mouthwash; and
wherein the composition is not substantially ingested.
